(19) United States
(12) Patent Application Publication
(10) Pub. No.: US 2026/0207893 A1
(43) Pub. Date: Jul. 23, 2026

(54) SYSTEMS, METHODS, AND DEVICES FOR CONTROLLING RE-SHEATHING FORCES

(71) Applicant: Cephea Valve Technologies, Inc., Abbott Park, IL (US)

(72) Inventors: Randolf von Oepen, Aptos, CA (US); Russell Patrick Shelton, San Jose, CA (US); Gregory Matthew Hyde, Menlo Park, CA (US)

(73) Assignee: Cephea Valve Technologies, Inc., Abbott Park, IL (US)

(21) Appl. No.: 19/465,065

(22) Filed: Jan. 30, 2026

Related U.S. Application Data
(63) Continuation of application No. 17/173,842, filed on Feb. 11, 2021, now abandoned.
(60) Provisional application No. 62/975,629, filed on Feb. 12, 2020.

(51) Int. Cl. A61M 25/01; A61F 2/24; A61M 25/00

(52) U.S. Cl. CPC A61M 25/0138; A61F 2/2436; A61M 25/0075; A61M 25/0136; A61M 25/0158

(57) ABSTRACT

A delivery system for delivering an interventional device to a targeted anatomical site includes an elongated delivery member having a proximal end and a distal end configured for housing the interventional device, and including a plurality of coaxially positioned delivery member components. The plurality of delivery member components including a delivery catheter having a bending portion having a coil section coaxially positioned with a braided portion and an inner catheter coaxially positioned within the delivery catheter and being adapted to maintain a connection with the interventional device until deployment of the interventional device. A handle assembly is provided for controlling movement of the delivery catheter and inner catheter.

### CROSS-REFERENCE TO RELATED APPLICATIONS

The pending application is a continuation of U.S. Patent Application Serial No. 17/173,842, filed Feb. 11, 2021, which application claims the benefit of and priority to U.S. Provisional Patent Application No. 62/975,629, filed Feb. 12, 2020, the disclosures of each of which are incorporated herein by this reference.

### BACKGROUND

### 1. Field of the Invention

The present disclosure generally relates to devices, systems, and methods for delivering an interventional device to targeted anatomy such as at the mitral annulus.

### 2. The Relevant Technology

Intravascular medical procedures allow the performance of therapeutic treatments in a variety of locations within a patient’s body while requiring only relatively small access incisions. An intravascular procedure may, for example, eliminate the need for open-heart surgery, reducing risks, costs, and time associated with an open-heart procedure. The intravascular procedure also enables faster recovery times with lower associated costs and risks of complication.

An example of an intravascular procedure that significantly reduces procedure and recovery time and cost over conventional open surgery is a heart valve replacement or repair procedure in which an artificial valve or valve repair device is guided to the heart through the patient’s vasculature. For example, a catheter is inserted into the patient’s vasculature and directed to the inferior vena cava. The catheter is then urged through the inferior vena cava toward the heart by applying force longitudinally to the catheter. Upon entering the heart from the inferior vena cava, the catheter enters the right atrium. The distal end of the catheter may be deflected by one or more deflecting mechanisms, which can be achieved by tension cable, or other mechanisms positioned inside the catheter. Precise control of the distal end of the catheter allows for more reliable and faster positioning of a medical device and/or implant and other improvements in the procedures.

An intravascularly delivered device needs to be placed precisely to ensure a correct positioning of the medical device, as the device may be difficult to reposition after the device is fully deployed from the delivery system. Additionally, the ability to recapture a partially deployed device is desirable in the event that the distal end of the catheter moves relative to the target location and compromises the precise positioning of the device.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates a delivery system configured for delivering, positioning, and deploying an interventional device, the delivery system including a handle assembly coupled to a delivery member;

FIG. 2 illustrates a cross-sectional view of the delivery member showing various delivery member components that may be utilized, including a steering catheter and a delivery catheter disposed within and translatable within the steering catheter;

FIG. 3 illustrates an exemplary approach for delivering an interventional device to the mitral annulus;

FIGS. 4A-7B illustrate various operations of the handle assembly to move components of the delivery member relative to one another;

FIG. 8 illustrates the outer sheath, showing various sections that may be formed in the outer sheath;

FIG. 9 is a cross-sectional view of the outer sheath of FIG. 8;

FIG. 10 is a partial cut-away view of an intermediate portion of the outer sheath;

FIG. 11 illustrates the steering catheter, showing various features and sections that may be formed in the steering catheter;

FIG. 12 illustrates the steering catheter after forming a compound curve shape to enable proper positioning of the delivery member relative to the mitral annulus;

FIG. 13A illustrates various cut patterns which may be utilized in the outer sheath, steering catheter, or delivery catheter, including the can structure, to provide flexibility and/or preferential bending;

FIG. 13B illustrates bending of the steering catheter, showing features of the cut patterns which enable the bending;

FIG. 14 illustrates a delivery catheter with distal cap structure configured for maintaining at least a portion of the interventional device in a compressed configuration pre-deployment;

FIG. 15 illustrates an exemplary suture catheter that may be disposed within the delivery catheter of FIG. 14 and which is configured for controlling axial tension on the replacement heart valve;

FIGS. 16A-16F illustrate deployment and release of the replacement heart valve at the mitral annulus;

FIG. 17 illustrates a coil section formed of a plurality of individual coil elements;

FIG. 18 illustrates a cross-sectional view of an example of an individual coil element;

FIG. 19 illustrates a cross-sectional view taken in parallel of a pair of holes on a coil element as shown in FIG. 18;

FIG. 20A illustrates a cross-sectional view of a coil section surrounded by a braid section;

FIG. 20B illustrates the cross-sectional view of FIG. 20A with the braided section moving relative to the coil section;

FIG. 20C illustrates a cross-sectional view of a coil section without a braided section with compression forces applied;

FIG. 20D illustrates a cross-sectional view of coil section surrounded by a braided section and an exemplary method of connecting a hypotube to the coil section;

FIG. 21 illustrates loops for controlling the collapse and subsequent opening of a ventricular anchor;

FIGS. 22A and 22B illustrate loops for controlling the collapse and subsequent opening of a ventricular anchor, the loops supporting various elongate member configurations;

FIGS. 23A and 23B illustrate cross-sectional views of an example configuration providing for the collapse and release of the ventricular disk; and

FIG. 24 illustrates a cross-sectional view of an example method of use of a retrieval catheter to recapture the valve.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers’ specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

Embodiments of the present disclosure solve one or more problems in the art with systems, methods, and devices for intravascular delivery of an interventional device to targeted intravascular anatomy, including a targeted cardiac valve. Suitable interventional devices that may be utilized in conjunction with the delivery system embodiments described herein may include valve repair devices, annuloplasty devices, valve clip devices, artificial heart valve devices, and other interventional devices. Embodiments described herein may be particularly useful for delivering interventional devices that move from a compressed, pre-deployed state to an expanded, deployed state.

### Delivery System Overview

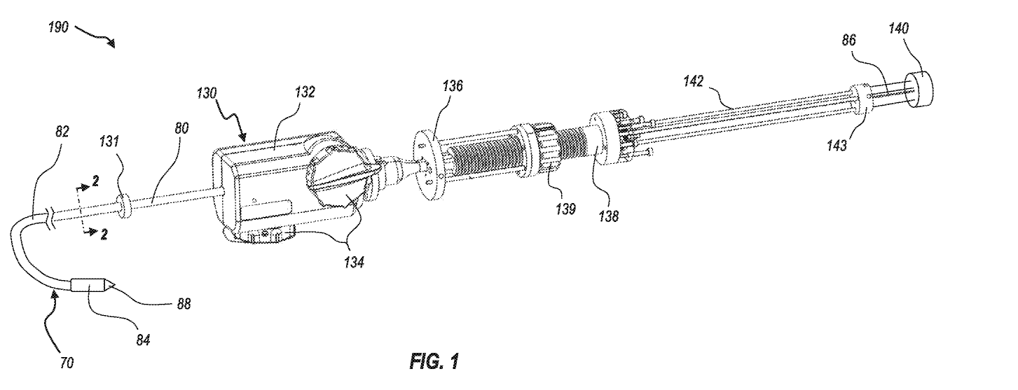

FIG. 1 illustrates an exemplary embodiment of a delivery system 190. As shown, the delivery system 190 includes a handle assembly 130 and a delivery member 70. The delivery member 70 is coupled to the handle assembly 130 and extends distally from the handle assembly 130. The delivery member 70 includes a plurality of catheter and/or lasercut hypotube members which provide different functionality during operation of the delivery system 190 to enable effective delivery and deployment of an interventional device.

The proximal end of an outer sheath 82 is coupled to an end ring 131, and the outer sheath 82 extends to a distal tip 88. A steering catheter handle 132 is disposed proximal of the end ring 131. The proximal end of a steering catheter 80 is coupled to the steering catheter handle 132, and the steering catheter 80 extends distally from the steering catheter handle 132 into the outer sheath 82. The steering catheter handle 132 includes one or more controls 134 which are operatively coupled to the steering catheter so that manipulation of the controls 134 adjusts the curvature of the steering catheter 80.

The outer sheath 82 extends to a distal end where it is coupled to a distal piece 84 (which may also be referred to herein as a “valve cover 84”). The distal piece 84 functions to house an interventional device in a compressed, pre-deployed state during intravascular delivery of the device to the targeted cardiac site.

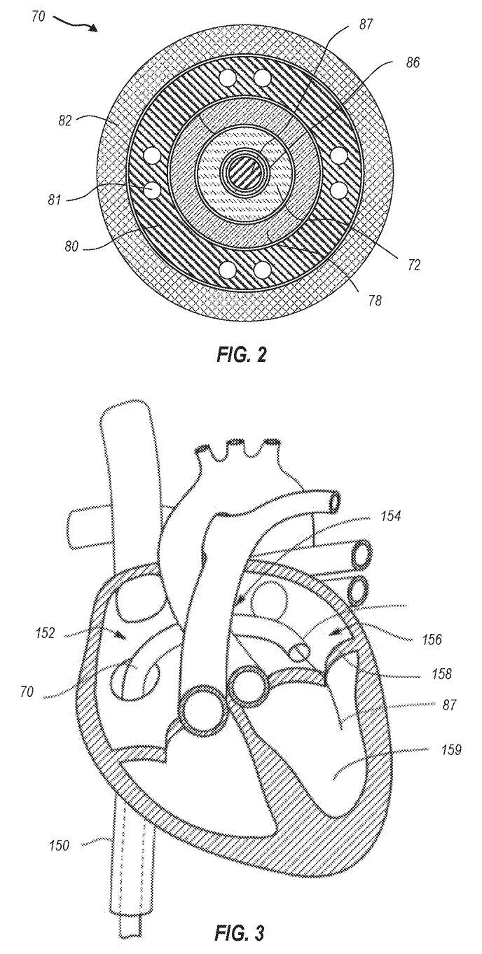

Because the steering catheter 80 is nested within the outer sheath 82, curving of the steering catheter 80 causes corresponding curving/steering in the outer sheath 82. The steering catheter 80 and outer sheath 82 may be referred to singly or collectively herein as the “outer member.” The illustrated embodiment of the delivery member 70 includes additional components which are not visible in the view of FIG. 1 but may be seen in the cross-sectional view of FIG. 2.

FIG. 2 illustrates a cross-sectional view of the delivery member 70 taken along the cross-section line 2-2. As shown, the steering catheter 80 is disposed within the outer sheath 82. A delivery catheter 78 (or alternatively referred to herein as an extension catheter) is disposed within the steering catheter 80. An inner catheter 72 (also referred to herein as suture catheter 72) may be disposed within the delivery catheter 78, and a guidewire tube 86 may be disposed within the inner catheter 72. The guidewire tube 86 is configured for receiving a guidewire 87. Although the particular nested configuration shown in FIG. 2 represents one preferred embodiment, alternative embodiments may include a different concentric arrangement of constituent parts. For example, some embodiments may combine the steering catheter 80 and outer sheath 82 and/or configure the outermost member with steering functionality, some embodiments may include more than one catheter with steering functionality, etcetera.

The steering catheter 80 is configured to be selectively curved to allow intravascular navigation. In some embodiments, the steering catheter 80 provides steerability via tension cables 83 received within a plurality of lumens 81 that extend through the length of the steering catheter 80. The lumens 81 may be configured for receiving tension cables 83 which extend between the controls 134 and the distal end of the steering catheter 80. One or more tension cables may additionally or alternatively be coupled to intermediate sections of the steering catheter 80. Manipulation of the controls 134 therefore adjusts tension in the tension cables to increase or decrease curvature of the steering catheter 80 at various positions. Although the controls 134 are shown here as knobs, alternative embodiments may additionally or alternatively include one or more buttons, sliders, ratcheting mechanisms, stepper motors, or other suitable controls capable of adjusting tension to provide steering. Illustrative structures that can be used as part of the steering catheter handle 132 and or steering catheter 80 are described in U.S. Patent No. 7,736,388, which is incorporated herein by this reference.

Referring to FIGS. 1 and 2, a delivery catheter holder 136 is disposed proximal of the steering catheter handle 132. Although not visible in the view of FIG. 1, the proximal end of the delivery catheter 78 is coupled to the delivery catheter holder 136. The delivery catheter 78 extends distally away from the delivery catheter holder 136 and into the steering catheter 80. An inner catheter holder 138 (also referred to herein as suture catheter holder 138) is disposed proximal of the delivery catheter holder 136. The inner catheter 72 may be coupled to the inner catheter holder 138 so that translation of the inner catheter holder 138 corresponds to translation of the inner catheter 72. For example, the inner catheter 72 may be selectively locked relative to the inner catheter holder 138 through a set screw, clamp, or other selective holding mechanism. The inner catheter 72 extends distally away from the inner catheter holder 138 and into the delivery catheter 78.

An inner catheter control 139 is operatively coupled to the inner catheter holder 138. Manipulation of the inner catheter control 139 adjusts the relative positioning of the delivery catheter holder 136 and inner catheter holder 138, and thus the relative positioning of the delivery catheter 78 and the inner catheter 72. In the illustrated embodiment, the inner catheter control 139 operates through threaded engagement with the inner catheter holder 138, such that rotation of the inner catheter control 139 translates the inner catheter holder 138 relative to the control 139 and therefore relative to the delivery catheter holder 136. Alternative embodiments may additionally or alternatively include one or more of a slider and rail assembly, a ratcheting mechanism, or other suitable means of linear adjustment.

The inner catheter 72 may extend proximally to and be attached to an inner catheter cap 143. A user may decouple the inner catheter 72 from the inner catheter holder 138 to allow movement of the inner catheter 72 by sliding/translating the inner catheter cap 143 along alignment rods 142. The guidewire tube 86 extends distally through the alignment cap 143 and into the inner catheter 72. The guidewire tube 86 extends to the distal end of the delivery member 70 where it is attached to a distal tip 88. The distal tip 88 is preferably formed from a flexible polymer material and provides a tapered, atraumatic shape which assists in passing the delivery member 70 through the vasculature and across the inter-atrial septum to the mitral annulus, which is required in a typical transfemoral approach to the mitral annulus.

In the illustrated embodiment, the guidewire tube 86 is coupled to a guidewire tube holder 140. By moving the guidewire tube holder 140, the guidewire tube 86 may be selectively translatable relative to the inner catheter cap 143 such that the guidewire tube 86 and distal tip 88 may be linearly translated relative to the inner catheter 72 and other components of the delivery member 70. The guidewire tube 86 may be selectively locked in longitudinal position relative to the inner catheter holder 138 and/or inner catheter cap 143, such as through a set screw, clamp, or other selective fastener. For example, such a fastening structure may be associated with the inner catheter cap 143.

When unlocked, the guidewire tube 86 (and likewise the distal tip 88) may be moved relative to the inner catheter 72. The ability to retract the distal tip 88 relative to the inner catheter 72 reduces the risk that the distal tip 88 will become overextended during deployment, where it could become tangled in chordae tendineae and/or cause injury to cardiac tissue. Additionally, independent movement of the guidewire tube 86 (with the distal tip 88) also allows for closing the gap between the distal tip 88 and the valve cover 84 following deployment of the intravascular device. When the intravascular device has been released, the distal tip 88 is separated from the valve cover 84 by a distance, such as by about 10 mm to about 100 mm, from about 20 mm to about 80 mm, from about 30 mm to about 50 mm, or about 40 mm. To avoid drawing air into the catheter, the gap between valve cover 84 and distal tip 88 is closed by drawing the distal tip 88 towards the valve cover 88, preferably in the left side of the heart, to avoid sucking air into the catheter when pulled back into the right side of the heart (where there is relatively low pressure).

FIG. 3 illustrates a schematic representation of a patient’s heart and a delivery procedure to the mitral annulus that may be conducted using the illustrated delivery system 190. The delivery member 70 may be inserted into the patient’s vasculature (e.g., through a transfemoral approach) and directed to the inferior vena cava 150. The delivery member 70 is passed through the inferior vena cava 150 toward the heart. Upon entering the heart from the inferior vena cava 150, the delivery member 70 enters the right atrium 152. For mitral valve related procedures, the delivery member 70 must further pass into the left atrium 156 by passing through a puncture in the intra-atrial septum 154. The puncture of the septum being performed prior to the insertion of the delivery system.

In other implementations, such as for procedures associated with a tricuspid valve, the delivery member 70 may be passed through the inferior vena cava 150 and into the right atrium 152, where it may then be positioned and used to perform the procedure related to the tricuspid valve. As described above, although many of the examples described herein relate to delivery to the mitral valve, one or more embodiments may be utilized in other cardiac procedures, including those involving the tricuspid valve.

Although a transfemoral approach for accessing a targeted cardiac valve is one preferred method, it will be understood that the embodiments described herein may also be utilized where alternative approaches are used. For example, embodiments described herein may be utilized in a transjugular approach, transapical approach, or other suitable approach to the targeted anatomy. For procedures related to the mitral valve or tricuspid valve, delivery of the replacement valve or other interventional device is preferably carried out from an atrial aspect (i.e., with the distal end of the delivery member 70 positioned within the atrium superior to the targeted valve). The illustrated embodiments are shown from such an atrial aspect. However, it will be understood that the interventional device embodiments described herein may also be delivered from a ventricular aspect.

In some embodiments, a guidewire 87 is utilized in conjunction with the delivery member 70. For example, the guidewire 87 (e.g., 0.014 in diameter, 0.018 in diameter, 0.035 in diameter) may be routed through the guidewire tube 86 of the delivery member 70 to the targeted cardiac valve.

Additional details regarding delivery systems and devices that may be utilized in conjunction with the components and features described herein are described in United States Patent Application Publication Numbers 2018/0028177A1 and 2018/0092744A1, which are incorporated herein by this reference.

### Operation of the Handle Assembly

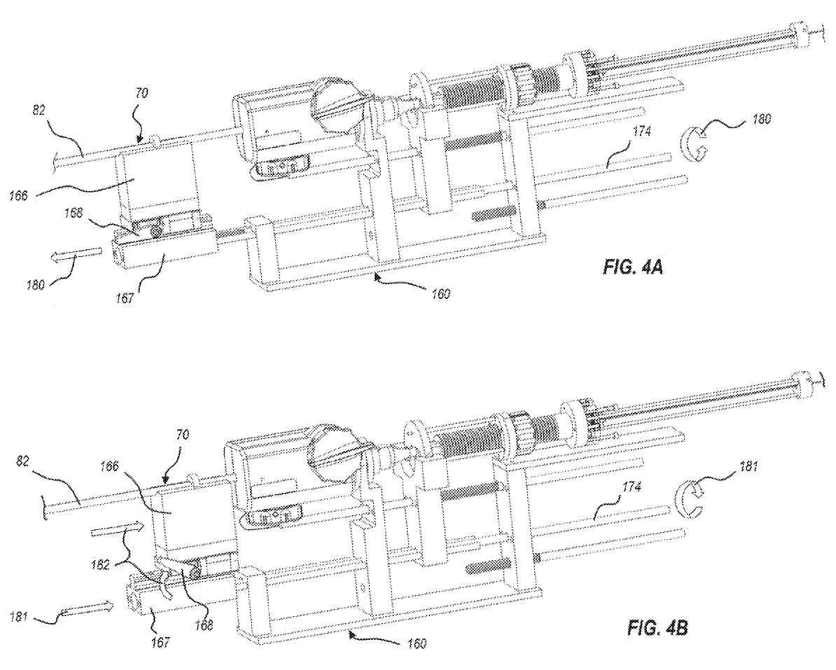

FIGS. 4A and 4B illustrate in greater detail operation of the handle assembly for translating the outer sheath 82. Sheath movement may be utilized to deploy an interventional device sheathed at or otherwise attached to the distal end of the outer sheath 82, or to recapture such an interventional device by advancing the outer sheath 82 over the device. The illustrated embodiment provides two modes for translating the outer sheath 82. The outer sheath adjustor 174 and the slider block 167 are coupled to each other with corresponding threads, and rotation of the outer sheath adjustor 174 causes the slider block 167 to translate. With the slider lock 168 engaged, the outer sheath support 166 and outer sheath 82 move with the slider block 167. The slider lock 168 may also be disengaged, allowing the outer sheath support 166 and outer sheath 82 to be manually advanced or retracted by sliding relative to the slider block 167.

As shown by corresponding arrows 180, rotation of the outer sheath adjustor 174 in one direction causes the slider block 167 to advance, and as shown by corresponding arrows 181, rotation of the outer sheath adjustor 174 in the opposite direction causes the slider block 167 to retract. In FIG. 4A, the slider lock 168 is in an engaged position. In FIG. 4B, arrows 182 show disengagement of the slider lock 168 and translation of the outer sheath support 166 upon the slider block 167. The dual mode adjustment of the outer sheath 82 beneficially allows a user to make different types of adjustments depending on procedural circumstances and/or preferences. For example, a user may make larger, quicker adjustments by unlocking the slider lock 168 and manually sliding the outer sheath support 166, and may make finer, more controlled adjustments by rotation of the outer sheath adjustor 174.

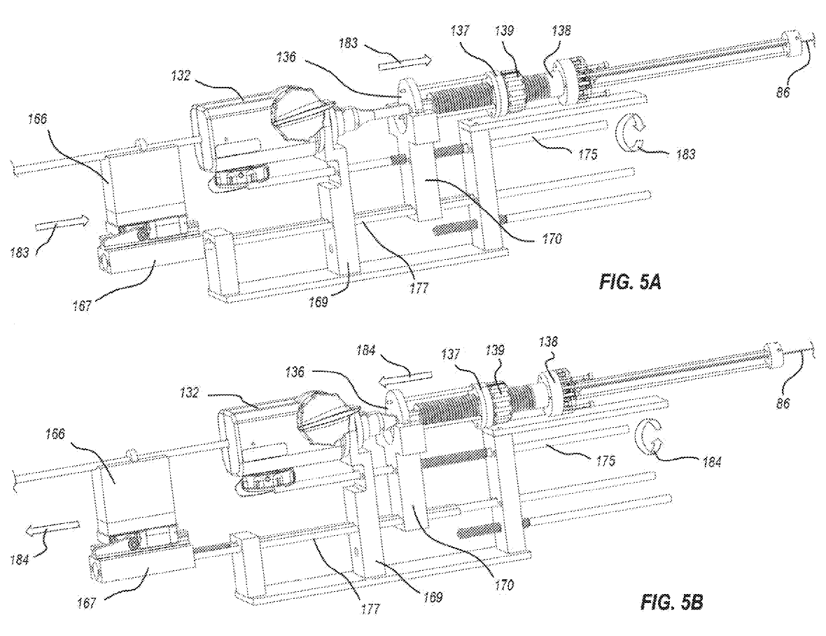

FIGS. 5A and 5B illustrate a deployment adjustment that moves several of the delivery member components relative to the steering catheter 80. FIG. 5A illustrates, by arrows 183, rotation of the deployment adjustor 175 in a first direction to retract the slider block 167, delivery catheter holder 136, and suture catheter holder 138. FIG. 5B illustrates, by arrows 184, rotation of the deployment adjustor 175 in a second direction to advance the slider block 167, deployment catheter holder 136, and suture catheter holder 138. As explained below, after the steering catheter 80 has been curved to orient the delivery member 70 with respect to the mitral annulus, the other components of the delivery member 70 can be advanced over the steering catheter 80 to move into a proper position for deployment of the interventional device. Holding the steering catheter 80 in place while the other components are advanced allows the compound curve of the steering catheter 80 to remain in the desired position.

The deployment adjustor 175 is threadedly engaged with the delivery catheter support 170. The connecting rods 177 mechanically link the delivery catheter support 170 to the slider block 167 to form a bracket assembly. The connecting rods 177 are able to freely pass through the steering catheter handle support 169 without engaging. The delivery catheter holder 136 and the suture catheter holder 138 are also mechanically linked as part of the bracket assembly by way of the alignment ring 137 and suture catheter control 139. Accordingly, rotation of the deployment adjustor 175 causes the delivery catheter holder 136, slider block 167, and suture catheter holder 138 to translate while the position of the steering catheter handle 132 is maintained. Translation of the outer sheath support 166 can be assured by locking to the slider block 167.

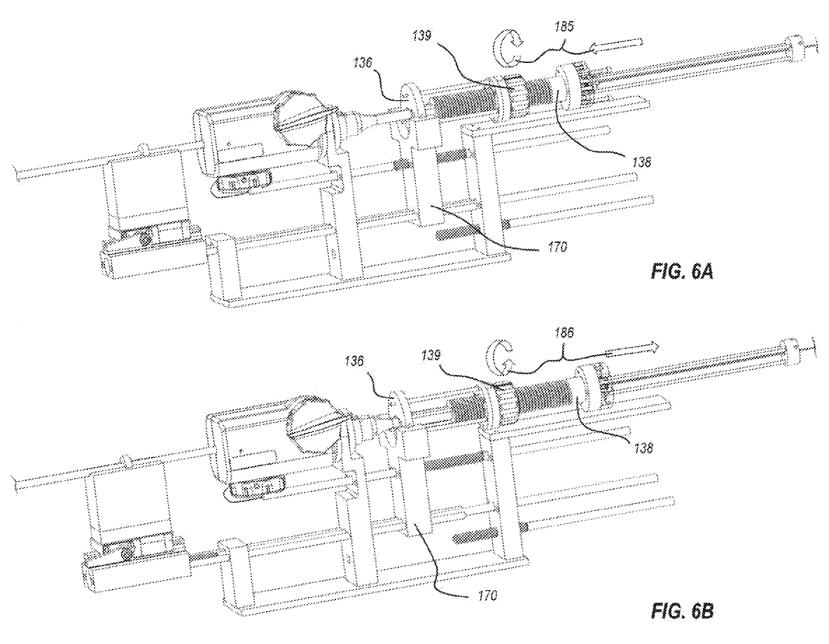

FIGS. 6A and 6B illustrate an operation for moving the suture catheter holder 138 relative to the delivery catheter holder 136. FIG. 6A shows, by arrows 185, that rotation of the suture catheter control 139 in a first direction causes the suture catheter holder 138 to advance relative to the delivery catheter holder 136. FIG. 6B shows, by arrows 186, that rotation of the suture catheter control 139 in a second direction causes the suture catheter holder 138 to retract relative to the delivery catheter holder 136. The threaded engagement of the suture catheter control 139 to the suture catheter holder 138 allows for finely controlled adjustments of the suture catheter position. As explained in more detail below, sutures of the suture catheter 72 (inner catheter 72) may be coupled to an interventional device while the device is in a pre-deployed state, and movement of the suture catheter 72 relative to the delivery catheter 78 allows tension of the sutures to be adjusted.

FIGS. 7A and 7B illustrate an operation for moving the guidewire tube 86 and guidewire tube holder 140 relative to the other components of the delivery member 70. FIG. 7A shows, by arrows 187, retraction of the guidewire tube holder 140 and corresponding retraction of the distal tip 88. FIG. 7B shows, by arrows 188 advancement of the guidewire tube holder 140 and corresponding advancement of the distal tip 88. The ability to adjust the distal tip 88 can lower the risk that the distal tip 88 undesirably interferes with chordae tendineae or other cardiac anatomy during deployment procedures. For example, during deployment of an interventional device, the suture catheter 72 may be advanced to disengage from the interventional device. If the distal tip 88 is not retracted relative to the advancing suture catheter 72, the distal tip 88 could extend too far into the ventricle where it could catch chordae tendineae and/or impinge against the cardiac wall.

### Elongated Delivery Member Components

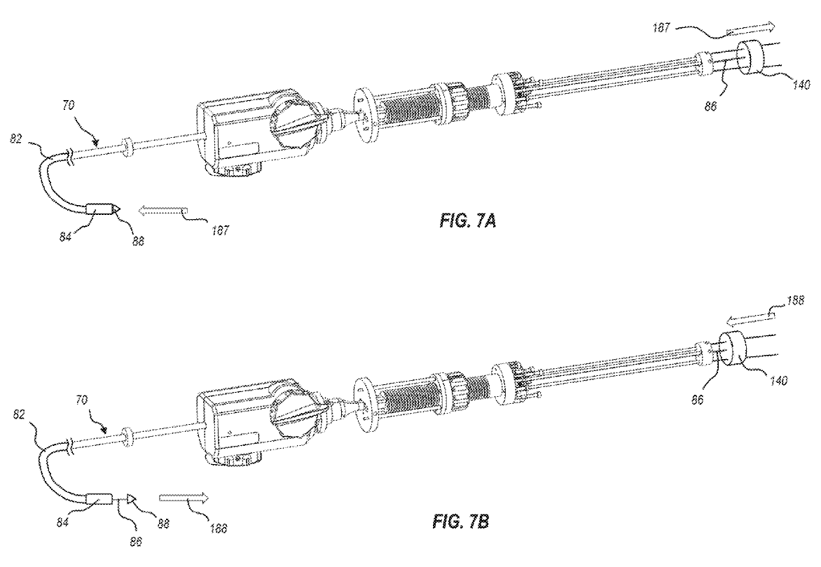

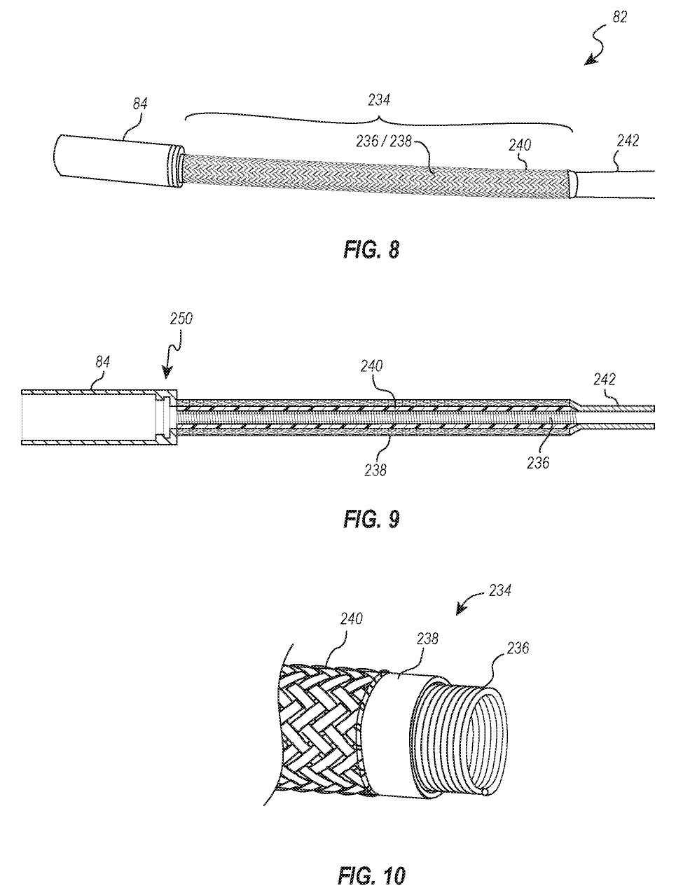

FIGS. 8 and 9 illustrate a portion of the distal end of the outer sheath 82 and distal piece 84 (also occasionally referred to herein as cover 84). Distal piece 84 can be formed as a cylindrical tube having an inner diameter and length sized to receive the interventional device, in a collapsed/pre-deployed configuration, within the lumen of distal piece 84. Distal piece 84 can include a plurality of microfabricated cuts (e.g., laser cuts) and a pair of continuous longitudinal spines located on opposite sides so that distal piece 84 can bend and flex substantially in a single plane. The outer sheath 82 can also include a bending portion 234 that can be attached to and located proximal to distal piece 84. Bending portion 234 may have a sufficient length to surround and extend along that portion of the delivery system that is designed to bend and reorient, via the steerable catheter 80, to navigate through a patient’s vasculature and/or heart to a target site for deploying the interventional device. In some embodiments, the bending portion 234 can include a cable tube or coil 236 surrounded by a braided structure 438 (sometimes collectively referred to as the “coil/braid portion 236/238”) as shown in FIG. 10.

Attached to the proximal end of bending portion 234 is a cut hypotube 242 that extends from bending portion 234 to the proximal end of the sheath 82. Hypotube 242 can include a plurality of slits and at least one longitudinally continuous spine that can preferably be continuous and uninterrupted along a longitudinal length of, and located at a fixed angular location on, hypotube 242.

In such embodiments, it can be desirable for the bending portion 234 of delivery catheter to remain liquid tight. To seal the bending portion 234, a flexible, fluid impermeable covering can be provided over the coil/braid portion 236/238, extending from the distal piece 84 to a location proximal the coil/braid portion 236/238. For example, the delivery sheath 82 can also include a thin walled flexible cover 240 that extends from the distal piece 84 to the hypotube 242. Flexible cover 240 can be bonded at each end to the underlying structure, using one of a variety of different adhesives, thermal adhesives, UV bonded adhesive, or other techniques.

Referring again to FIG. 9, outer sheath 82 can also be coupled to distal piece 84 via a swivel connection, generally indicated at 250. To overcome the challenging forces that can develop during insertion of a relatively large delivery catheter into the vasculature of a patient, swivel connection 250 allows rotation of outer sheath 82 by a few degrees, back and forth (i.e., alternating between clockwise rotation and counter-clockwise rotation) while at the same time moving the delivery system 200 in a generally longitudinal direction. This rotational motion (during simultaneous longitudinal translation) helps to overcome some of the longitudinal forces, such as friction, such as static friction, that may resist insertion of outer sheath 82 through a patient’s vasculature or frictional forces between the outer sheath 82 and the steering catheter 80.

FIG. 11 illustrates one embodiment of the steering catheter 80 in greater detail. In the illustrated embodiments, the steering catheter 80 includes a proximal section 318, intermediate section 316, and a distal section 314. A steering ring 510 (also referred to herein as a tip ring) is connected at the distal end. The one or more tension cables, described above, may extend through the steering catheter 80 and engage with or attach to one or more steering ring(s) 310 to provide manipulation and control of the curvature of the steering catheter 80. A distal cap 312 positioned over the steering ring 310 or integrally formed with the steering ring 310 provides an angled/rounded surface that allows the steering catheter 80 to more effectively move and slide against the outer sheath 82 without binding. In this embodiment, the steering catheter 80 is formed as a hypotube, such as a laser cut hypotube. The proximal section 518 may remain uncut, while the intermediate section 316 and distal section 314 may be cut (e.g., laser cut) to increase flexibility. Although not shown in this view, a polymer layer may surround the steering catheter and forms an outer layer.

In some embodiments, the steering catheter 80 is rotationally keyed to the outer sheath 82. The outer sheath 82 may include cut patterns and/or other features which are arranged to provide particular preferred bending directions. In this embodiment, because bending of the outer sheath 82 depends upon curving of the steering catheter 80, rotational alignment of the outer sheath 82 to the steering catheter 80 is beneficial. These components may be keyed together using a key and corresponding keyway feature, slots and corresponding tabs, or other rotational keying mechanism known in the art. Alternatively, or additionally, alignment markers can be provided at the handle assembly to visually indicate alignment.

To provide effective steering and positioning at the mitral annulus, the distal section 314 is cut with a pattern which allows a bending radius of about 15 mm or less (e.g., 5 to 15 mm). The intermediate section 316 is cut to allow a bending radius of about 30 to 45 cm. The proximal section is uncut to provide the steering catheter 80 with sufficient stiffness, torquability, and pushability and also the ability to flush the system.

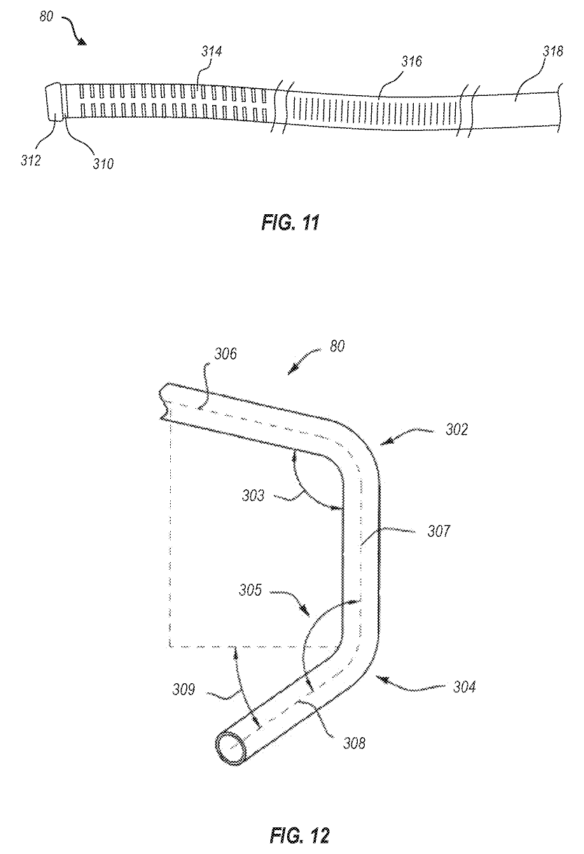

FIG. 12 illustrates an example of a series of compound bends that the steering catheter 80 may perform during the delivery, repair, recapture, or repositioning of the interventional device. While accessing the mitral annulus, the steering catheter 80 may be steered in at least two planes of motion. The two planes of motion may be substantially perpendicular to one another. The steering catheter 80 has a first bend 302 with a first bend angle 303 measured between a first longitudinal axis 306 and a second longitudinal axis 307. In some embodiments, the first bend angle 303 may be in a range of about 40° to about 120°, more often about 90° to about 120°, or about 105°. A second bend 304 is formed between a third longitudinal axis 308 and the second longitudinal axis 307. The second bend 304 may also have a rotational angle 309 relative to a plane in which the first longitudinal axis 306 and the second longitudinal axis 307 lie. In other words, the rotational angle 309 is relative to the amount of rotation of the third longitudinal axis 308 relative to the direction of the first bend 302. In one embodiment, the second bend angle 305 is in a range of about 45° to 135° or about 60°.

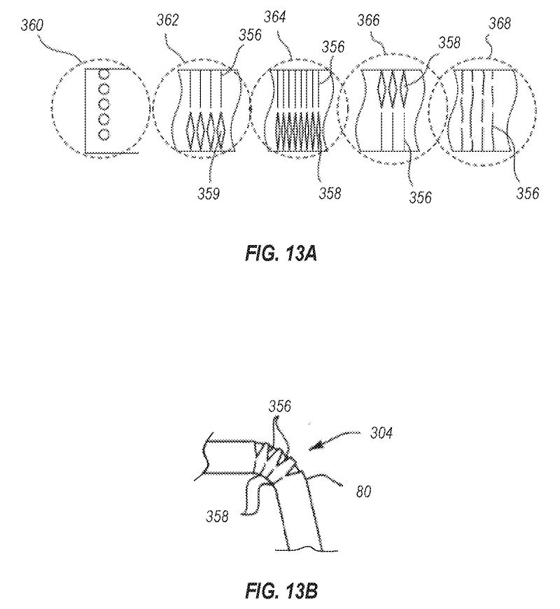

FIG. 13A shows various cutting patterns that can be used in different sections of the steering catheter 80 (and corresponding sections of the outer sheath 82) to produce the desired bends; it being understood that the different cutting patterns can be disposed at any location(s) along a length of the steering catheter 80 and the outer sheath 82, including the distal piece 84. Additionally, and more generally, the various cutting patterns of FIG. 13A can be used on any structures of the delivery system to provide desired flexibility, pushability, and/or torqueability. Each section can include cut patterns that can include one or more slits 356 and/or one or more island cuts 358. The slits 356 may transmit longitudinal force along the catheter and also allow expansion of the catheter when it is deflected in a direction opposite the slit 356. The island cuts 358 may allow compression of the catheter when it is deflected in a direction of the island cuts 358. For example, slits 356 and island cuts 358, when located on opposite sides from one another, may direct preferential bending of the catheter, as shown by exemplary bend 304 in FIG. 13B.

In one embodiment, illustrated in FIG. 13A, a cutting pattern can include five sections or regions 360, 362, 364, 366 and 368, with different cut patterns in each section. Such sections may be arranged as needed to provide the desired compound curve profile. For example, a first section 360 can include a plurality of holes radially spaced about the periphery of the catheter. A second section 362 provides for bending in a first direction, a third section 364 is similar to the second section 362 but with smaller sized and more closely spaced island cuts 358, a fourth section 364 provides for bending in a second direction, and a fifth section 366 includes multiple slits for adding flexibility without forming a particular bending direction. While the island cuts 358 are depicted as diamond-shaped, the island cuts 358 may have one or more other shapes, such as square, rhombohedral, triangular, rectangular, circular, oblong, other elliptical, other polygonal, irregular, or combinations thereof.

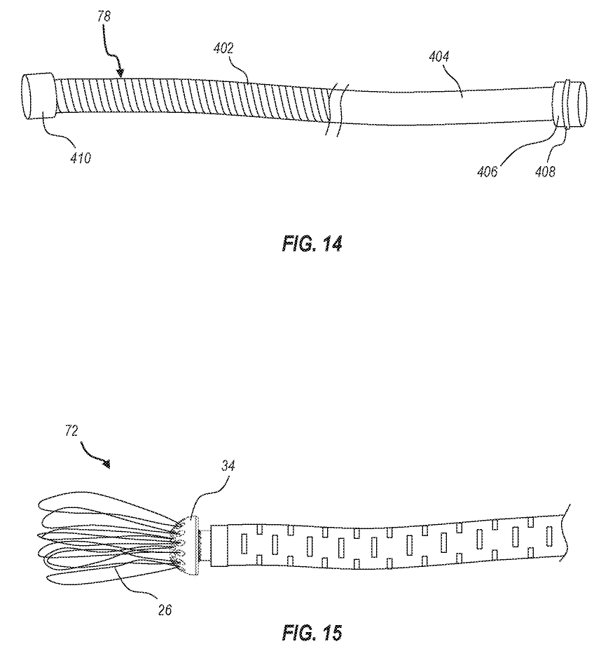

FIG. 14 illustrates one embodiment of the delivery catheter 78. The delivery catheter 78 includes a proximal section 404 and a distal section 402. At the proximal end, the delivery catheter 78 may include a seal 406 and an O-ring 408 for forming a fluid tight seal at the handle assembly 130, in particular at the delivery catheter holder 136. In the illustrated embodiment, the distal section 402 is formed as a coil. The coil provides the delivery catheter 78 with ability to effectively push the valve device through the steering catheter 80 as part of deploying the valve device. The coil also provides good flexibility for navigating a patient’s tortuous vasculature.

The delivery catheter 78 also includes a can structure 410 disposed at the distal end. The can 410 is configured to constrain and hold at least a proximal section of a collapsible/expandable interventional device 10. Without such constraint, the outer portion of the device 10 may bias radially outward against the inner surface of the overlying components of the delivery member 70, making it more difficult to unsheathe or re-sheathe the device 10.

The can 410 may also have a length sufficient to aid in maintaining coaxial alignment of the distal end of the delivery catheter 78 within the delivery member 70 to avoid or minimize unwanted tilting. For example, the can 410 preferably has a length to diameter ratio of greater than or equal to 1, though in alternative embodiments the ratio may be smaller, such as about 0.25 to 1, depending on the stiffness of the distal section 402. In still other configurations, the can 410 can take the form of a plate and so have no depth. In some The can 610 also provides an effective structural surface to act as a counterforce to maintain the interventional device 10 in the proper pre-deployed position when the outer member is retracted. In some embodiments, one or more edge portions of the can 410 include a taper and/or smooth surface for easier sliding of the can 410 within the outer member.

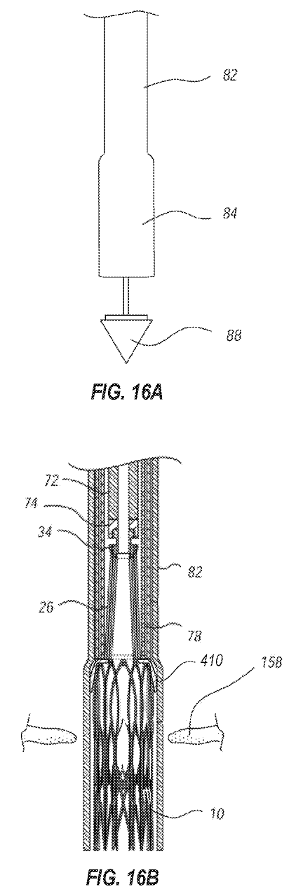

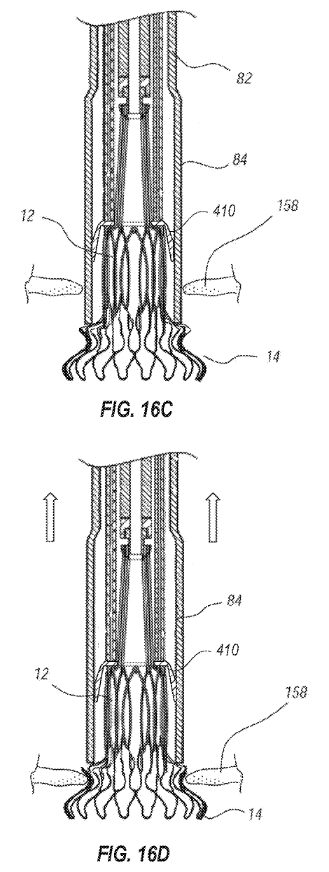

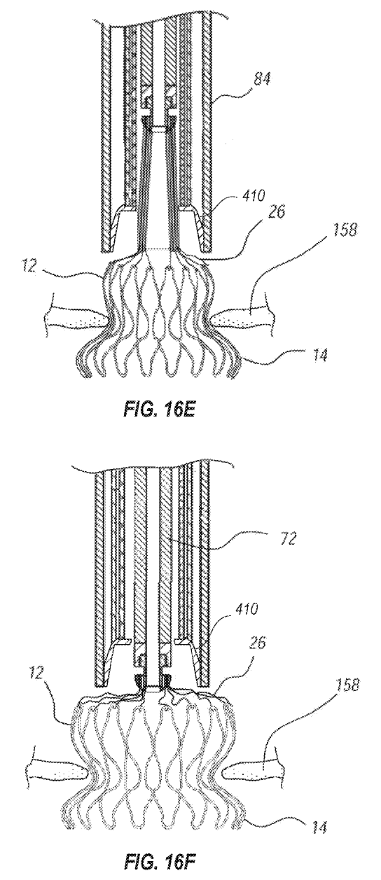

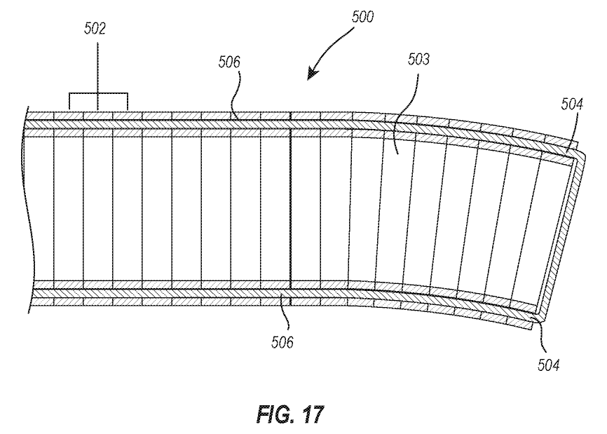

FIG. 15 is a detail view of the inner catheter 72 (which may also be referred to herein as a “suture catheter”). The inner catheter 72 may be utilized as part of the delivery member 70 to maintain axial tension of the interventional device prior to deployment, and by so doing may aid in maintaining at least the proximal section of the interventional device within the can 410. For example, the inner catheter 72 may include a connecting ring 34 with a series of suture loops 26 that may be tethered to corresponding attachment points of the interventional device (see further details in FIGS. 16A-16F). Retraction of the inner catheter 72 relative to the delivery catheter 78 adds axial tension to the interventional device to maintain it in a pre-deployed position while distal movement of the inner catheter 72 relative to the delivery catheter 78 releases axial tension and allows deployment of the device.

### Deployment of the Interventional Device

FIGS. 16A-16F schematically illustrate deployment and release of an interventional device 10 (shown here as a replacement valve) at the mitral annulus 158. As shown in FIG. 16A, the distal tip 88 is first advanced relative to the outer sheath 82 and valve cover 84 to provide sufficient space for deployment. For clarity, in following Figures, the tip 88 is not shown. FIG. 16B shows in cross-section the delivery member in position at the mitral annulus 158, with a distal portion of the valve 10 positioned on the ventricular side, and a proximal portion of the valve 10 positioned on the atrial side. Partial retraction of the outer sheath 82, as shown in FIG. 16C, allows the ventricular anchor 14 to release and expand. As shown in FIG. 16D, the valve 10 may then be retracted proximally to bring the ventricular anchor 14 into contract against the mitral annulus 158, thus optionally engaging one or more hooks, barbs, or other structures into vascular tissue, such as the mitral valve. This may be accomplished by retracting the delivery catheter 78. Alternatively, the entire delivery member 70 may be retracted.

As shown by FIG. 16E, the valve cover 84 may then be further retracted to release the atrial anchor 12 on the atrial side of the mitral annulus 158. At this point, the valve 10 may still held by the suture loops 26 in a position not yet fully deployed. This allows the valve 10 to be further positioned or recaptured if necessary. As shown in FIG. 16F, the suture catheter 72 may then be distally advanced to relieve tension in the suture loops 26, allowing the atrial anchor 12 to more fully expand and release from the can structure. Even further distal advancement of the delivery catheter detaches the suture loops 26 and allows the delivery member 70 to be removed from the patient. The longitudinal position of the tip 88 relative to the suture catheter 72 can be adjusted as needed while the suture catheter 72 is advanced. After the valve 10 is detached, the tip 88 is retracted and reconnected to the valve cover 84 prior to removal of the delivery member from the patient.

Additional details related to coils structures, coil braid structures, collapsing of the valve, and retrieval of the valve will now be presented. It will be understood that any of the structures, methods and functional aspects of the coil structures, coil braid structures, methods and structures for collapsing of the valve, and methods and structures for retrieving the valve can be used with or combined with any other structure, methods, apparatuses, and functional aspects described herein.

### Coil Structure

To achieve high forces when re-sheathing a valve, a coil can be both flexible and withstand high compression forces without collapsing. In general, stacked coils can be somewhat flexible, but if the thickness of the coil is limited, they can tend to collapse under high compression, such that single coils shift transversely to a longitudinal axis of the coil as a whole. This can occur, such as when the coil is not constrained on either the inside diameter (ID) or outside diameter (OD) of the coil.

To overcome this limitation, a coil section 500 can be formed of a plurality individual coil elements 502 that are mounted on elongate members 504, such as wires, sutures, or other elongate members running through holes 506 of the individual coil elements 502, as illustrated in the FIG. 17. The coil element 502 can comprise a body 503 in a substantially ring-like shape. The individual coil elements 502 can be aligned by a series of elongate members 504 extending through the coil elements 502. As illustrated, the elongate members 504 can extend along holes 506 and then return along other holes 506 when the elongate members 504 are not attached or connected to the last coil element 502. For instance, when there are pairs of holes 506, the elongate members 504 can extend through the holes 506 of the pair and then return through the other holes 506 of the pair. Alternatively, the end of the elongate members 504 can be fixed to the last coil element 502, such as through welding, chemical or mechanical bonds, fasteners, etc.

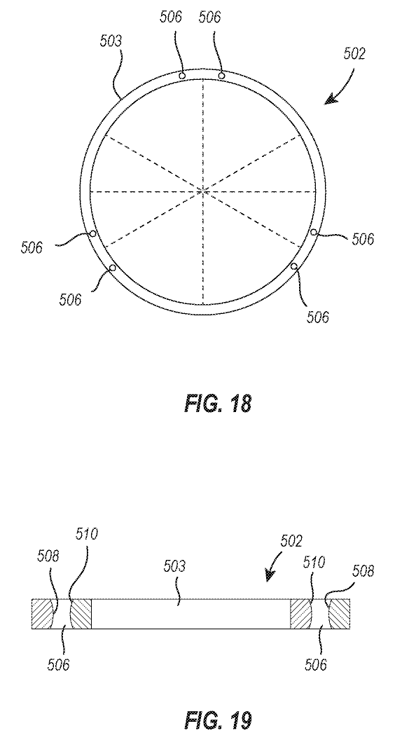

Illustrated in FIG. 18, one example of a coil element 502 having a thickness of about 0.5mm to about 1.5mm, with an OD of about 0.395” and an ID of about 0.365.” As shown, the coil element 502 has 3 pairs of holes 506 which are offset by about 120 degrees, the individual holes each having a diameter of about 0.007”. It will be understood, however, that the dimensions, number of hole pairs, angular offset, and hole diameter can vary based upon the particular configuration of the coil element.

For instance, there can be less than or greater than 3 pairs of holes 506 formed in the coil elements 502 and so the angular offset can be greater than or less than about 120 degrees. The number of pairs of holes 506 can be about 2 pairs of holes 26 to about 12 pairs of holes 506, from about 3 pairs of holes 506 to about 9 pairs of holes 506, from about 4 pairs of holes 506 to about 8 pairs of holes 506, or some other grouping of pairs. The angular offset can range from about 30 degrees to about 180 degrees, from about 40 degrees to about 120 degrees, from about 45 degrees to about 90 degrees, or another angular offset. The diameter of the holes 2006 can be greater than or less than the 0.0007”. The elongate member 504 extending through the holes 506 can have a diameter of about 0.006” or such other size to allow it to move within the hole 506 based upon the hole’s diameter. The elongate member 504 can be formed of a metal, alloy, polymer, composite, combinations or modifications thereof. The material forming the elongate member 504 can be selected based upon a desired maximum or minimum elongate. For instance, if the elongate member 504 is formed of a shape-memory materials, such as NITINOL, the elongate member 504 can have an elongation of less than about 8%. It will be understood that the elongation can be less than or greater than 8% based upon the particular material chosen. For instance, the desired elongate can be from about 2% to about 10%, from about 4% to about 8%, or some other elongation.

The individual coil element 502 can be formed of stainless steel. In another configuration, the individual coil elements 502 can be formed of Polyetheretherketone (PEEK). Alternatively, the coil element 502 can be formed of a metal, alloy, composite, polymer, ceramic, combinations of modifications thereof.

As mentioned above, the holes 506 can receive the elongate members 504. The holes 506 can have walls 508 that are generally parallel, taper from one side to the other in a longitudinal axis or direction of the hole 506, form an hour-glass shape in cross-section taken parallel to the longitudinal axis, or some other cross-section. The configuration illustrated in FIG. 19 has generally tapered holes 506 that provide for smooth movement of the elongate member 504 through the hole 506 during bending. The tapered or chamfered sides 530 prevent catching or damage to the moving elongate member 504. The holes 506 can be formed using various machining techniques, such as a short pulse laser, etc.

### Coil Braid Structure

During deployment of an implantable medical device, such as the interventional device 10 described in relation to FIGS. 16A-16F, the interventional device or valve 10 is under high compressive forces within the valve cover 82. The compressive forces result from collapsing the ventricular and arterial disks or anchors 12 and 14 of the valve 10 from a diameter of about 50 mm to a diameter of about 11 mm or less. Collapsing the valve 10 to fit within the valve cover 82 can require sheathing forces of about 25 pounds to about 150 pounds, from about 40 pounds to about 100 pounds, or about 50 pounds to about 100 pounds. To achieve these forces at a distal end of the catheter can be challenging, particularly when flexibility and steerability are needed to deflect the distal tip of the catheter, including the valve cover 84 and the sheathed valve 10, in two planes.

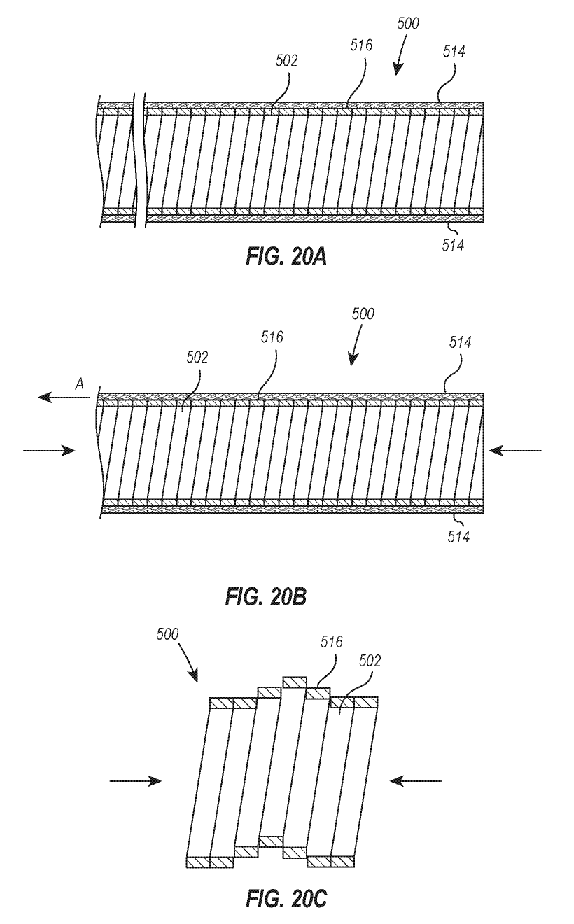

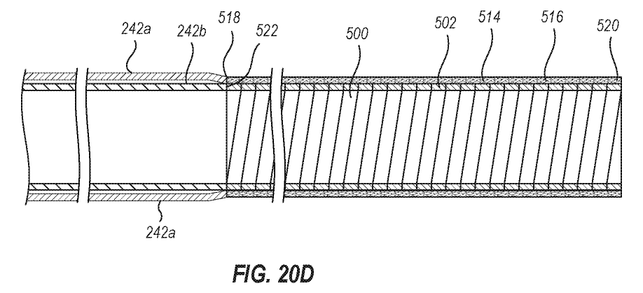

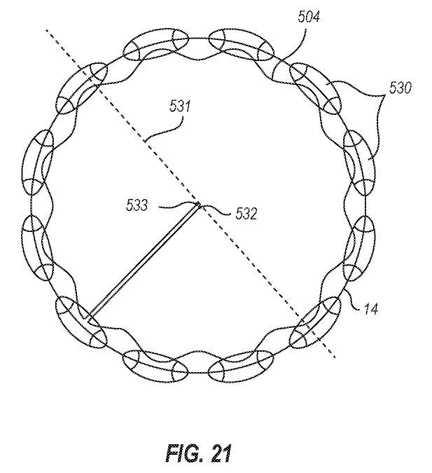

While the bending port 234 of the described outer sheath 82 of FIG. 8, for instance, can include a coil 236 surrounded by the braid 238, the following combination of braid section 534 and coil section 500 (see FIGS. 20A and 20B) includes a fixed distal end, with movable proximal ends of the braid section 534 and coil section 500. This allows the braid section 534 and the coil section 500 to be independent actuated during unsheathing or re-sheathing.

Turning to FIG. 20A, the coil section 500 and braid section 534 are operated independently from each other. The braid section 534, for instance, is only connected at a distal section of the catheter, such as at the distal end of the coil through soldering, welding, adhesives, etc. When the valve 10 is unsheathed, a pull force A can be applied to the braid section 534 thereby locking or forcing the braid section 534 against an outer surface 536 of the coil section 500, which can be a stacked coil with substantially no or little space between the individual coil elements 502.

In contrast to unsheathing the valve 10, when re-sheathing the valve, compressive forces are applied to the coil section 500. The coil section 500 still provides flexibility to movement and positioning of the valve cover 84. During re-sheathing, to avoid the offsetting of the coil elements 502 as described above and illustrated in FIG. 20B, a pull force A is applied to the braid section 534 to constrain the coil elements 502, as illustrated in FIG. 20C. This constraint prevents or limits coil element 502 movement, thereby allowing the coil section 500 to transmit the high compressive forces without individual coil elements 502 becoming offset.

As illustrated in FIG. 20D, a proximal end of the braid section can be connected to a distal end of a first hypotube 242a or other tubular structure, such as through soldering, welding, adhesives, etc. Similarly, a proximal end 522 of the coil section 500 can be connected to a distal end of a second hypotube 242b or other tubular structure, such as through soldering, welding, adhesives, etc. These hypotubes 242 can be moved relative to each other to create the desired locking and relative movement.

With this configuration, a catheter can accommodate high tension during unsheathing and high compression during re-sheathing, while maintaining flexibility to move the catheter, including the valve cover 84 in 1, 2, or more planes.

### Controlling Valve Collapse and Expansion

During deployment of an implantable medical device, such as the interventional device 10 described in relation to FIGS. 16A-16F, the interventional device or valve 10 is under high compressive forces within the valve cover 82. The compressive forces result from collapsing the ventricular and arterial disks or anchors 12 and 14 of the valve 10 from a diameter of about 50 mm to a diameter of about 11 mm or less. Because of the high compressive forces it can be desirable to control expansion of the valve 10 as it is deployed from the valve cover 82, such as controlling deployment of the ventricular anchor 14 so that it can be completely or partially deployed or moved from the collapsed state. This controlled deployment allows for repositioning the valve 10 during deployment and implantation. The valve 10 requires anchoring forces to ensure that the valve 10 will stay in position after it is placed. To ensure adequate anchoring, different anchoring mechanisms can be used such as radial force, pinching the annulus between two disks, hook, and other mechanism.

In one configuration, to enable collapsing of the valve, a funnel can be used to help the valve collapse more easily. Furthermore, the collapse of the valve is normally done below the AF temperature of NITINOL which can significantly reduce the force on the valve 10. Nevertheless, even when using a funnel under low temperatures, the sheathing forces can be around 25lbs, 40lbs, 50lbs, 100lbs, 150lbs or more.

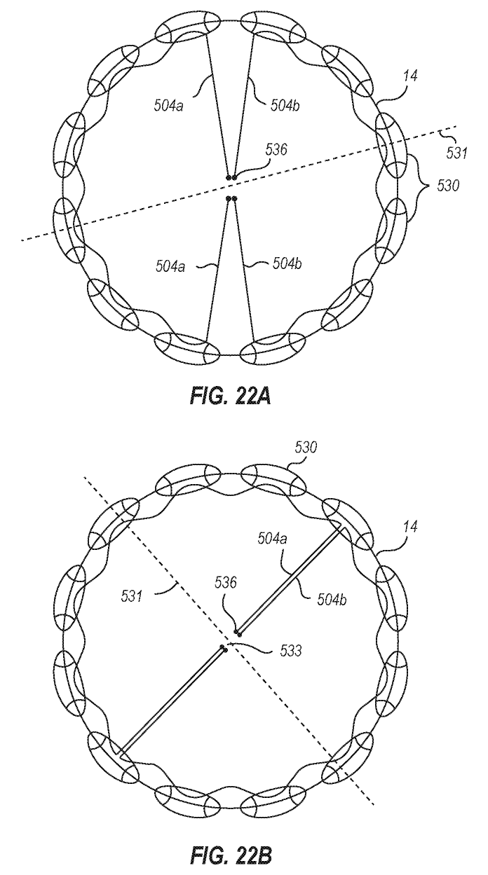

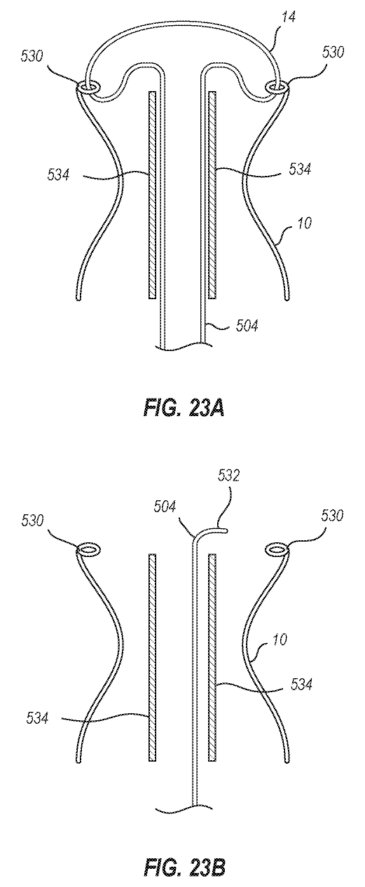

To control the collapse, and subsequent opening, of the ventricular anchor 14, loops 530 are formed between adjacent crests, as illustrated in FIG. 21. The loops 530 can be formed of suture or other material, such as members formed of metal, alloy, polymer, composite, natural or synthetic material, combinations or modifications thereof. This allows for control of the two crests at the same time since the ventricular anchor 14 can include more crests than the arterial anchor 12, such as having double the number of crests. While a twelve-loop configuration is shown in FIGS. 21-22B, it will be understood that any number of loops 530 are possible, such as less than twelve, greater than twelve, etc.

The loops 530 can be a guide for an elongate member 504, such as a NITINOL wire or a suture, that can draw the loops 530 towards a central axis 531 of the ventricular anchor 14 and to allow for symmetrical collapse of the ventricular anchor 14. For instance, as illustrated in FIG. 21, a single elongate member 504 can be threaded through the loops 530 with both ends 532 of the elongate member 504 exiting through a center 533 of the ventricular anchor 14 toward the handle assembly 130. When tension is applied to the ends 532 of the elongate member 504 by longitudinal, rotational, and/or a combination of both longitudinal and rotational movement of the inner catheter 72 (FIGS. 16A and 16F) or other portion of the delivery catheter 190, the loops 530 slide along the elongate member 504 to collapse the ventricular anchor 14.

Alternatively, two elongate members 504a and 504b can be threaded through different groupings of the loops 530 as illustrated in FIG. 22A. The elongate members 504a and 504b can be threaded through apertures 536 which can be located on the central axis 531 or adjacent to the central axis 531, as shown in FIGS. 22A and 22B. In still another configuration, two elongate members 504a and 504b can be threaded through different groupings of the loops 530, with one or more of the loops 530 being used by each of the elongate members 504a and 504b at the same time, as illustrated in FIG. 22B.

While reference is made to using two elongate members 504a and 504b, it will be understood that more than two elongate members 504a and 504b can used with an associated number of grouping of the loops 530. Additionally, while reference is made to the loops 530 extending between two crests, it will be understood that the loops 530 can be associated with a single crest or more than two crests.

In one configuration of the catheter described herein, a spacer or balloon can be used during sheathing the valve 10 into the valve cover 10 to avoid non-uniform folding of the valve 10. While the spacer or balloon can be removed following sheathing, the space formed by the spacer or balloon can be used to provide a channel for the elongate members 504 described herein. For instance, a compression resistant element 534 can be disposed at the center of the valve 10, as illustrated in FIGS. 23A and 23B, and protrude slightly beyond at least one of a proximal end and distal end of the valve 10. The compression resistant element 534 can be a stacked coil, cable tube, laser cut hypotube, braided polymer shaft, helical hollow strand tube, or other tubular structures and have an ID of about 4 mm (it being understood that the ID can be greater or lesser than 4 mm, such as about 1 mm to about 7mm, from about 2 mm to about 6 mm, or about 3 mm to about 5 mm).

As illustrated in FIGS. 23A and 23B, the valve 10 includes the loops 530 attached to the ventricular anchor 14. In the center of the valve 10 is the compression resistant flexible tube 534 that is positioned protruding through the plane of the ventricular anchor 14 or the inner structure of the valve 10. The elongate member 504 is threaded through all the loops 530 and guided through the center of the compression resistant tube 534. When the elongate member 504 is pulled back, the loop formed by the elongate member 504 passing through the loops 530 closes to collapse the ventricular anchor 14.

To release the ventricular anchor 14, one end 532 of the elongate member 504 is released and the other end pulled towards the handle, such as one of the handles of the handle assembly 130, until the elongate member 504 has passed through all loops 530 and is withdrawn into or toward the center of the compression resistant tube 534, as illustrated in FIG. 23B.

### Implant Retrieval Catheter

In some circumstances, it is beneficial to retrieve the interventional device, such as the valve 10. This can be achieved while maintaining control of the valve 10 that is attached to the suture catheter 72 (FIG. 15) through the suture loops 26 (FIG. 15). Tension can be applied to the elongate member 504 passing through the loops 530 associated with the ventricular anchor 14 to at least partially collapse the ventricular anchor 14, such as by applying tension to the elongate member 504 by the inner catheter 72 (FIGS. 16A and 16F) or other portion of the delivery catheter 190. By collapsing the ventricular anchor 14, any barbs or hooks associated with the ventricular anchor 14 can be detached from vascular tissue. The collapsing of the ventricular anchor 14 can result in the ventricular anchor 14 having a diameter of about 20 mm to about 25 mm, or smaller. The disengagement of the ventricular anchor 14, and at least partially collapsing to a reduced diameter, not only allows for retraction of the ventricular anchor 14, and the valve 10 as a whole, into the left atrium 156 for retrieval, but also allows for repositioning and subsequent deployment of the ventricular anchor 14 as desired by the user.

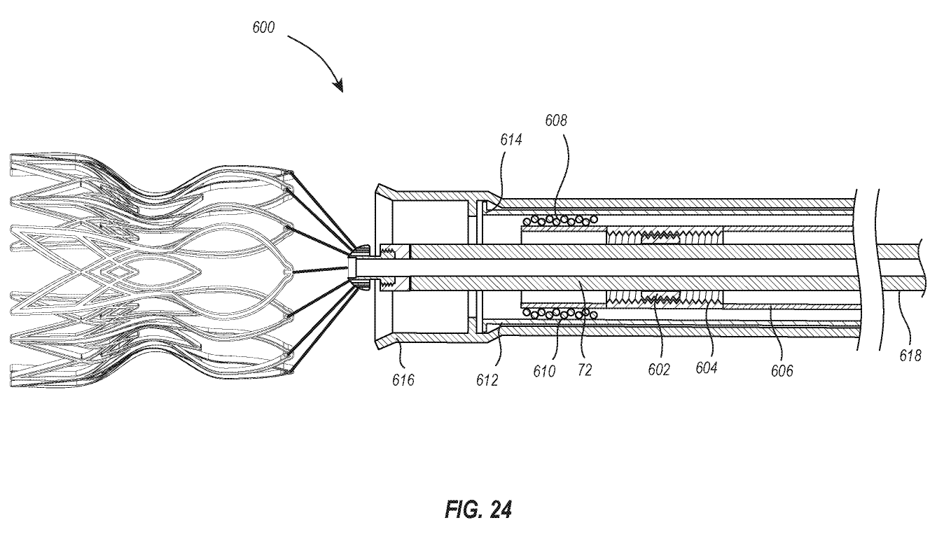

With the ventricular anchor 14, and the valve 10 as a whole, pulled into the left atrium 156 there is a relatively straight line from the suture catheter (72) extending through the vena cava 150 into the left atrium 156. This allows the valve 10 to be re-sheathed without articulation of a distal section of a retrieval catheter 600 (FIG. 24) thereby reducing the forces needed to collapse the valve 10 sufficiently to be received within the retrieval catheter 600 and eliminating higher force application if the suture catheter 600 is deflected in multiple planes.

FIG. 24 illustrates the orientation of the retrieval catheter 600 in relation to the valve 10 after the delivery catheter 78, the steering catheter 80, and the outer sheath 82 are removed thereby leaving the suture catheter 72 in place ad ready for capturing of the valve 10 by the retrieval catheter 500. FIG. 24 does not illustrate the distal tip 88 (FIG. 16A) and the associated guidewire tube 86 (FIG. 2) that extends to the distal tip 88 through the connecting ring 34.

Removal of the delivery catheter 78, the steering catheter 80, and the outer sheath 82 can be achieved following positioning at least the ventricular anchor 14 of the valve 10 in the left atrium 156 (FIG. 3). With the suture catheter 72 detached from a stabilizer, an extension 618 can be threaded, friction fit, clamped, or otherwise connected to the proximal end of the suture catheter 72 to increase a length of the suture catheter 72. This combination of extension 618 and suture catheter 72 act as a rail along which a remainder of the delivery system 190 (FIG. 1) can be removed. For instance, the addition length provided by the extension 618 allows the user to first hold the suture catheter 72 in position close to the patient’s leg and after the suture catheter 72 is extending though a retrieval catheter 600. With suture catheter 72 under tension by the user controlling or manipulating the proximal end of the suture catheter 72, the retrieval catheter 600 can be advanced to capture or retrieve the valve 10.

With the ventricular anchor 14, and the valve 10 as a whole, pulled into the left atrium 156 there is a relatively straight line from the suture catheter 72 extending through the vena cava 150 into the left atrium 156. The retrieval catheter 600 is advanced along the suture catheter 72 and into the left atrium 156 so that the valve 10 is captured within the retrieval catheter 72. Following re-sheathing or capture, the retrieval catheter 600, the valve 10, and the suture catheter 72 can be removed. This process, with the straight line from the suture catheter 72 extending through the vena cava 150 into the left atrium 156, allows the valve 10 to be re-sheathed without articulation of a distal section of the retrieval catheter 600, thereby reducing the forces need to collapse the valve 10 sufficiently to be received within the retrieval catheter 600 and eliminating higher force application if the suture catheter 600 is deflected in multiple planes.

While it is possible to advance the retrieval catheter 600 towards the distal end of the suture catheter 72, to overcome the collapse forces of about 25 pounds to about 150 pounds, about 40 pounds to about 100 pounds, or about 50lbs to about 100, associated with re-sheathing or collapsing the valve 10 into the retrieval catheter 600, a threaded engagement can be used where rotational movement is converted into longitudinal movement of the retrieval catheter 600 along the suture catheter 72. To achieve this movement, the suture catheter 72 can include a threaded portion 602 that engages with a threaded portion 604 on a catheter 606. The threaded portion 602 provides an anchor point close to the end of the catheter.

Disposed at a distal end of the catheter 606 are protrusions 608 that can mate with complementary protrusions 610 formed in catheter 612 in a driving engagement where rotational movement of the catheter 612 is translated into longitudinal movement or displacement of the catheter 612 in relation to the catheter 606. More generally, an outside diameter or surface of the catheter 606 can mate with an inside diameter or surface of the catheter 612. When the catheter 612 is rotated, it moves forward relative to the catheter 606, while being fixed to the suture catheter 72. The catheter 612 can move forward or backward along the catheter 606 to advance an outer catheter 614; a distal end 614 of the catheter 612 being keyed to the outer catheter 616 so that the outer catheter 616 can be advanced but not rotated during rotation of the catheter 612.

The rotational or screwing mechanism from the protrusions 608 and 610 can be used to transmit high forces to collapse the valve 10 into a cavity formed at a distal end or cover of the outer catheter 616. Optionally, a distal end of the outer catheter 616 can include guiding feature to aid with retrieval of the valve 10. The guiding feature can include a tapered portion of the cover, rounded edges, or other structures to aid with retrieval of the valve 10.

One or more of the catheters 600, 606, 612 can be a hollow shaft, such as a hypotube, a helical strand shaft, or other structure having a lumen. The protrusions 608 and 610 can be formed by welding or joining one or more elements, such as coil elements to the catheters 606 and 612.

### Further Example Embodiments

Following are some further example embodiments of the invention. These are presented only by way of example and are not intended to limit the scope of the invention in any way.

Embodiment 1. A delivery system for delivering an interventional device to a targeted anatomical site, the delivery system including an elongated delivery member having a proximal end and a distal end configured for housing the interventional device, and including a plurality of coaxially positioned delivery member components, the plurality of delivery member components including a delivery catheter having a bending portion having a coil section coaxially positioned with a braided portion, an inner catheter coaxially positioned within the delivery catheter and being adapted to maintain a connection with the interventional device until deployment of the interventional device, and a handle assembly for controlling movement of the delivery catheter and inner catheter.

Embodiment 2. The delivery system of embodiment 1, further including an interventional device formed from nitinol configured to be housed within an outer sheath of the elongated delivery member.

Embodiment 3. The delivery system of any of embodiments 1-2, wherein the coiled section comprises a plurality of coil elements with at least one elongate member extending through the plurality of coil elements.

Embodiment 4. The delivery system of any of embodiments 1-3, wherein each coil element comprises a plurality of holes organized in pairs circumferentially around the coil element.

Embodiment 5. The delivery system of any of embodiments 1-4, wherein each hole has walls that are parallel, tapered, or form an hour-glass shape in a cross-section.

Embodiment 6. The delivery system of any of embodiments 1-5, wherein the coil section is a stacked coil with substantially no space between adjacent coil elements.

Embodiment 7. The delivery system of any of embodiments 1-6, wherein a distal end of the braid section and a distal end of the coil section are fixed relative to a distal end of the delivery catheter.

Embodiment 8. The delivery system of any of embodiments 1-7, wherein a proximal end of the braid section is mounted to a first movable tubular member and a proximal end of the coil section is mount to a second movable tubular member, the first movable tubular member being movable independently from the second movable tubular member.

Embodiment 9. The delivery system of any of embodiments 1-8, wherein the braid section is selectively locked against an outer surface of the coil section.

Embodiment 10. The delivery system of any of embodiments 1-9, wherein the interventional device comprises at least two loops.

Embodiment 11. The delivery system of any of embodiments 1-10, further comprising an elongate member extending through the at least two loops, the elongate member selectively moveable to draw the at least two loops centrally to collapse at least a portion of the interventional device.

Embodiment 12. The delivery system of any of embodiments 1-11, wherein the elongate member extends through a center of the interventional device.

Embodiment 13. The delivery system of any of embodiments 1-12, wherein the elongate member extends through a compression resistant tube disposed within the interventional device.

Embodiment 14. A system for delivering an interventional device to a targeted anatomical site, the delivery system including an elongated delivery member having a proximal end and a distal end configured for housing the interventional device, and including a plurality of coaxially positioned delivery member components, the plurality of delivery member components including a delivery catheter having a bending portion, an inner catheter coaxially positioned within the delivery catheter and being adapted to maintain a connection with the interventional device until deployment of the interventional device, the inner catheter comprising a threaded portion at a location proximal a distal end of the inner catheter, and a handle assembly for controlling movement of the delivery catheter and inner catheter.

Embodiment 15. The system of embodiment 14, further comprising a retrieval catheter selectively mounted to the inner catheter when the delivery catheter is removed from coaxial positioning around the inner catheter.

Embodiment 16. The system of any of embodiments 14-15, wherein the retrieval catheter includes a first catheter comprising a threaded portion complementary to the threaded portion of the inner catheter and a second catheter disposed on the first catheter and being rotatable in relation to the first catheter.

Embodiment 17. The system of any of embodiments 14-16, wherein the first catheter and the second catheter are drivingly engaged so that rotational movement of the second catheter longitudinally displaces the second catheter in relation to the first catheter.

Embodiment 18. The system of any of embodiments 14-17, wherein a distal end of the second catheter has a keyed relationship with an outer catheter in that rotational movement of the second catheter is not translated to the outer catheter.

Embodiment 19. The system of any of embodiments 14-18, wherein the outer catheter comprising a cavity configured to receive the interventional device.

Embodiment 20. The system of any of embodiments 14-19, wherein the cavity is formed in a valve cover having guiding feature to aid with retrieval of the interventional device.

### Conclusion

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

What is claimed is:

1. A method of deploying a prosthetic heart valve, comprising:
navigating a delivery system through a vasculature of a patient and to a native valve annulus, a sheath of the delivery system being configured to house the prosthetic heart valve in a compressed state while navigating the delivery system through the vasculature;
moving a catheter of the delivery system to control deployment of a ventricular end of the prosthetic heart valve by releasing tension on a strand that i) has a first portion configured to extend radially outward from a compression resistant member, ii) is configured to couple to the prosthetic heart valve and configured to extend around a circumference of the ventricular end of the prosthetic heart valve, and iii) has a second portion configured to extend radially inward to the compression resistant member; and
moving the catheter of the delivery system to control deployment of an atrial end of the prosthetic heart valve.

2. The method of claim 1, wherein moving the catheter of the delivery system to control deployment of the ventricular end of the prosthetic heart valve comprises: longitudinal movement of the catheter; rotational movement of the catheter; or a combination of longitudinal and rotational movement of the catheter.

3. The method of claim 1, wherein the catheter is an inner catheter disposed within at least one of: an outer sheath of the delivery system, or a steering catheter of the delivery system.

4. The method of claim 3, wherein the inner catheter is a suture catheter, and moving the suture catheter of the delivery system to control deployment of the atrial end comprises distally advancing the suture catheter to relieve tension in a plurality of suture loops coupled to the atrial end of the prosthetic heart valve.

5. The method of claim 3, wherein the delivery system comprises a handle having an inner catheter control, wherein manipulation of the inner catheter control adjusts a position of the inner catheter.

6. The method of claim 1, wherein:
the first portion extends radially outward from a distal end of the compression resistant member; and
the second portion extends radially inward to distal end of the compression resistant member.

7. The method of claim 1, wherein:
the first portion emerges and extends radially outward from an open distal end of the compression resistant member; and
the second portion extends radially inward and through the open distal end of the compression resistant member.

8. The method of claim 1, wherein the strand is coupled to the prosthetic heart valve and extends around the circumference of the ventricular end of the prosthetic heart valve by passing through a plurality of loops of the ventricular end of the prosthetic heart valve.

9. The method of claim 8, wherein the strand passes through each of the plurality of loops of the ventricular end.

10. The method of claim 8, wherein the strand passes through the plurality of loops to form a strand loop, and controlling deployment of the ventricular end of the prosthetic heart valve further comprises tensioning the strand and tensioning the strand causes the strand loop to close and collapse the ventricular end of the prosthetic heart valve.

11. The method of claim 1, wherein moving the catheter of the delivery system to control deployment of the ventricular end occurs prior to moving the catheter of the delivery system to control deployment of the atrial end.

12. The method of claim 1, wherein the compression resistant member comprises one of: a stacked coil; a cable tube; a laser cut hypotube; a braided polymer shaft; or a helical hollow strand tube.

13. The method of claim 1, wherein the compression resistant member is configured to be positioned in a center of the prosthetic heart valve and protrudes through a plane of the ventricular end of the prosthetic heart valve.

14. The method of claim 1, wherein the strand is threaded through a central lumen of the compression resistant member.

15. The method of claim 1, wherein the strand is a Nitinol wire or a suture.

16. The method of claim 1, wherein controlling deployment of the ventricular end of the prosthetic heart valve occurs as the prosthetic heart valve is deployed from the sheath.

17. The method of claim 1, wherein the sheath is a valve cover configured to advance or retract relative to the prosthetic heart valve.

18. The method of claim 1, further comprising:
retracting the sheath to expose the ventricular end of the prosthetic heart valve.

19. The method of claim 1, wherein controlling deployment of the ventricular end of the prosthetic heart valve comprises partially deploying the ventricular end.

20. The method of claim 19, wherein controlling deployment of the ventricular end of the prosthetic heart valve comprises repositioning the prosthetic heart valve during controlled deployment of the ventricular end of the prosthetic heart valve.
